(19) United States
(12) Patent Application Publication
(10) Pub. No.: US 2014/0249425 A1
(43) Pub. Date: Sep. 4, 2014

(54) Method of Determining Affirmative and Negative Response Areas in a Human Cerebral Cortex

(71) Applicant: Ming-Ta Wu, Taipei City (TW)

(72) Inventor: Ming-Ta Wu, Taipei City (TW)

(21) Appl. No.: 14/273,802

(22) Filed: May 9, 2014

Related U.S. Application Data
(63) Continuation-in-part of application No. 13/206,404, filed on Aug. 9, 2011, now abandoned.

(51) Int. Cl. A61B 5/026; A61B 5/16

(52) U.S. Cl. CPC A61B 5/0261; A61B 5/164
USPC 600/473

(57) ABSTRACT

A method of determining affirmative and negative response areas in a cerebral cortex of a human subject under a test, comprises (A) providing a testing apparatus to detect real-time variations in cerebral blood flow, (B) generating a test array consisting of a plurality of test points in a tested area of the subject's head to detect real-time variations of the cerebral blood flow in the tested area, wherein the tested area approximately corresponds to an affirmative or negative response area in the cortex; (C) asking a question to the human subject, wherein the question is designed so that an answer for the question is either yes or no; and (D) determining a precise position of the affirmative or negative response area in the cortex, according to an active region corresponding to the real-time variations of the cerebral blood flow generated in 3 seconds within the tested area after answering.

### CROSS REFERENCE TO RELATED APPLICATIONS

This application is a continuation-in-part of U.S. patent application Ser. No. 13/206,404, filed on Aug. 9, 2011, which is incorporated herewith by reference.

### BACKGROUND OF THE INVENTION

1. Field of the Invention

The present invention relates to a method of determining affirmative and negative response areas in a cerebral cortex by detecting dynamic variations in the cerebral blood flow.

2. The Prior Arts

Lie detection techniques usually combine psychology (in particular psychology and physiology), criminology, criminal investigation, electronics, and other related techniques. Questions are usually designed to measure physiological changes triggered by emotional stimulation. The lie detector may typically measure variation in respiration, heartbeat, blood pressure, and muscular tension. Presently, most lie detectors can include respiration measure apparatuses, galvanic skin response detectors, and blood pressure measure apparatuses.

The conventional lie detectors described above are limited to measures of physiologic responses to detect lies. However, lies are usually the result of brain reflection activities. Therefore, lie detection should be conducted by using a system that can measure the active state of the human cortex in order to yield precise determination. Currently, frequently used apparatuses for measuring the brain state include electroencephalogram detectors and functional magnetic resonance imaging (FMRI) apparatuses. Electroencephalogram detectors can only detect brain waves, whereas the FMRI apparatus is substantially large in volume and cannot provide convenient use. Moreover, the FMRI apparatus cannot provide real-time responses as to whether the human subject under test is lying. On the other hand, near infrared reflectance spectroscopy (NIRS) is a technique that can detect light reflectance occurring in the subject's head to measure variations in the blood flow that occur in the cortex. NIRS technique is not affected by electromagnetic noise, and can provide high resolution. Moreover, technical progress in NIRS allows providing real-time variations in the cerebral blood flow.

In the previous studies, Tian et al. (2009) disclose a deception detection model using fNIRS to investigate hemodynamic responses to deception in PFC (prefrontal cortex), and the results indicates that deception response is highly associated with PFC region. In another study, Monti et al. (2010) disclose a model for willful modulation of brain activity in disorders of consciousness disorders. They used functional magnetic resonance imaging (fMRI) to assess each patient's ability to generate willful, neuroanatomically specific, blood-oxygenation-level-dependent responses during two established mental-imagery tasks.

However, Infrared light (800-1000 nm) can penetrate to a depth of about 15-20 mm in maximum for detection of human hemodynamic response. As shown in Yuich et al. (2003), an adult head model is segmented into five types of tissue: scalp, skull, CSF, gray matter, and white matter (p. 2882, right column; FIG. 1a). Due to the limitation of penetration depth of near infrared light (15-20 mm) for human tissues, the penetration of the spatial sensitivity profile into the white matter is scarcely observed (p. 2885, left column; FIG. 4a-4d). Similarly, SHIMADZU (one of the largest NIRS manufacturers) website (http://www.shimadzu.com/an/lifescience/imaging/nirs/nirs2.html) also indicates that near-infrared light can penetrate at a distance about 20 mm deep from the surface of the head, which can be used for detection of a change in oxygenated hemoglobin concentration.

Lie detection using NIRS mainly relies on detecting whether the affirmative and negative response areas are active to determine if the subject's response to a question is the truth. However, the actual positions of the affirmative and negative response areas may differ among different individuals. Accordingly, it may be necessary to precisely determine the positions of the affirmative and negative response areas in the cortex so that the lie detection method applied subsequently can provide more accurate results.

### SUMMARY OF THE INVENTION

An objective of the present invention is to provide a method of determining affirmative and negative response areas in the cortex of a human subject. According to the present invention, the method comprises:
(A) providing a testing apparatus to detect real-time variations in cerebral blood flow;
(B) generating a test array consisting of a plurality of test points in a tested area of the subject's head to detect real-time variations of the cerebral blood flow in the tested area, wherein the tested area corresponds to an affirmative or negative response area in the cortex;
(C) asking a question to the human subject, wherein the question is designed so that an answer for the question is either yes or no; and
(D) determining a precise position of the affirmative or negative response area in the cortex according to an active region corresponding to the real-time variations of the cerebral blood flow generated in 3 seconds within the tested area, after answering;
   wherein the testing apparatus is a near-infrared spectroscopy apparatus; asking of step (C) and answering of step (D) are performed in oral form; when the answer is “yes” in step (C), the position determined in step (D) is a affirmative response area; when the answer is “no” in step (C), the position determined in step (D) is a negative response area; the affirmative response area and the negative response area are located at left parietal lobe and right parietal lobe respectively, the affirmative response area is located in a region of Cz-Fz-F7-T3 defined by international 10-20 system, and the negative response area is located in a region of Cz-Fz-F8-T4 defined by international 10-20 system.

With the method of the present invention, the affirmative and negative response areas of each individual can be precisely determined, which allows to improve the accuracy of the results provided by the lie detection applied afterwards.

### BRIEF DESCRIPTION OF THE DRAWINGS

The present invention will be apparent to those skilled in the art by reading the following detailed description of preferred embodiments thereof, with reference to the attached drawings, in which:

FIG. 1 is a flowchart of a method according to the present invention;

FIG. 2 is a schematic view showing a method of determining a negative response area in the cortex;

FIG. 3 is a schematic view showing a method of determining an affirmative response area in the cortex;

FIG. 4 is a schematic view showing an example of actual testing results to determine a negative response area in a human cortex;

FIG. 5 is a schematic view showing an example of actual testing results to determine an affirmative response area in a human cortex; and

FIGS. 6A and 6B demonstrate the affirmative response area and the negative response area in a human cerebral cortex with 10-20 system.

### DETAILED DESCRIPTION OF THE PREFERRED EMBODIMENTS

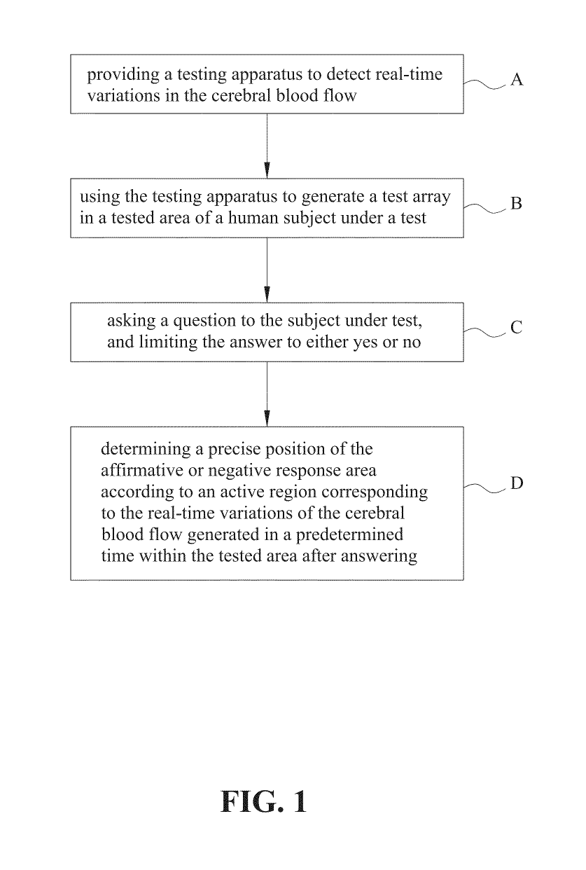

FIG. 1 is a flowchart showing a method of determining affirmative and negative response areas in a human cortex.

Step (A): provide a testing apparatus to detect real-time variations in cerebral blood flow. In this embodiment, a near-infrared spectroscopy apparatus for cerebral blood flow detection applying near-infrared reflectance spectroscopy (NIRS) can be used to detect light irradiation reflected from the human's head and accordingly derive changes in the cerebral blood flow. This technique is not affected by electromagnetic noise, which can provide high resolution measures. In particular, the used testing apparatus can show real-time variations in the cerebral blood flow.

Step (B): the testing device is used to generate a test array consisting of a plurality of test points in a tested area of the subject's head. This test array can be used to detect real-time variations of the cerebral blood flow in the tested area. The tested area corresponds to the affirmative or negative response area in the cortex. This step has to be performed after the position of the area to be tested is properly determined.

Step (C): ask a question to the human subject under test, and require the subject to answer by “yes” or “no”. The question can be based on personal information related to the tested subject (for example the gender, the age, or other background information whose correct answers are known in advance).

Step (D): once the tested subject has answered the question, the position of the affirmative or negative response area in the subject's cortex is accurately determined as the active region in the tested area where real-time variations of the cerebral blood flow occur within a predetermined time. Preferably, the determination conducted in this step can be made based on real-time variations of the cerebral blood flow in the tested area within 1 second, 2 seconds or 3 seconds after the question is answered. If this time is too short, the variation in the cerebral blood flow may not be correctly reflected; in case this time interval is too long, the variation in the cerebral blood flow may have already finished. Accordingly, the time interval cannot be excessively short or long.

### Embodiment 1: Determination of the Negative Response Area in the Cortex

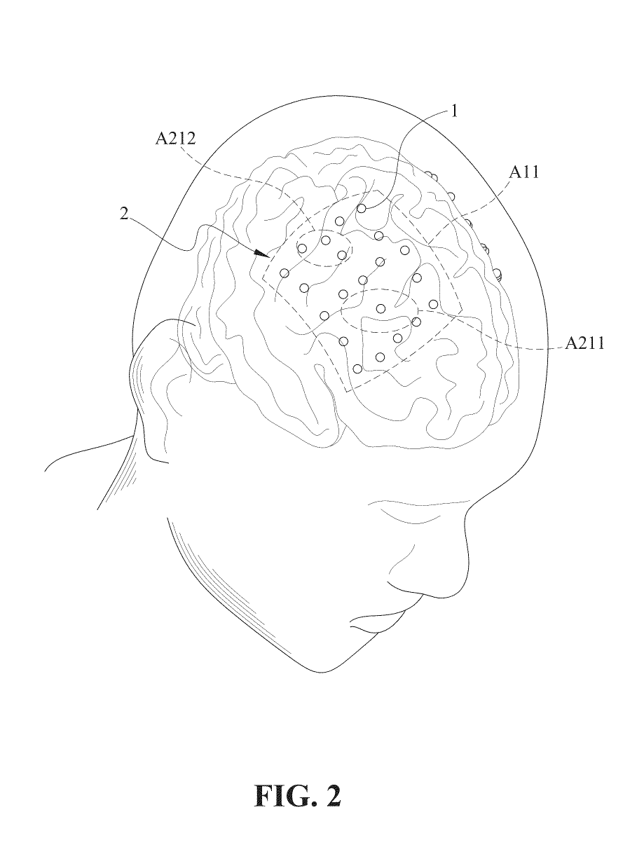

FIG. 2 is a schematic view showing a method of determining a negative response area in the cortex. The steps of this method are similar to the aforementioned method. After it is properly set, the near-infrared spectroscopy apparatus for cerebral blood flow detection can be used to generate a test array 2 consisting of a plurality of test points 1 in a tested area All of the subject's head. As shown in FIG. 2, the test array respectively comprises light emitting points needed for the testing, and light detecting points.

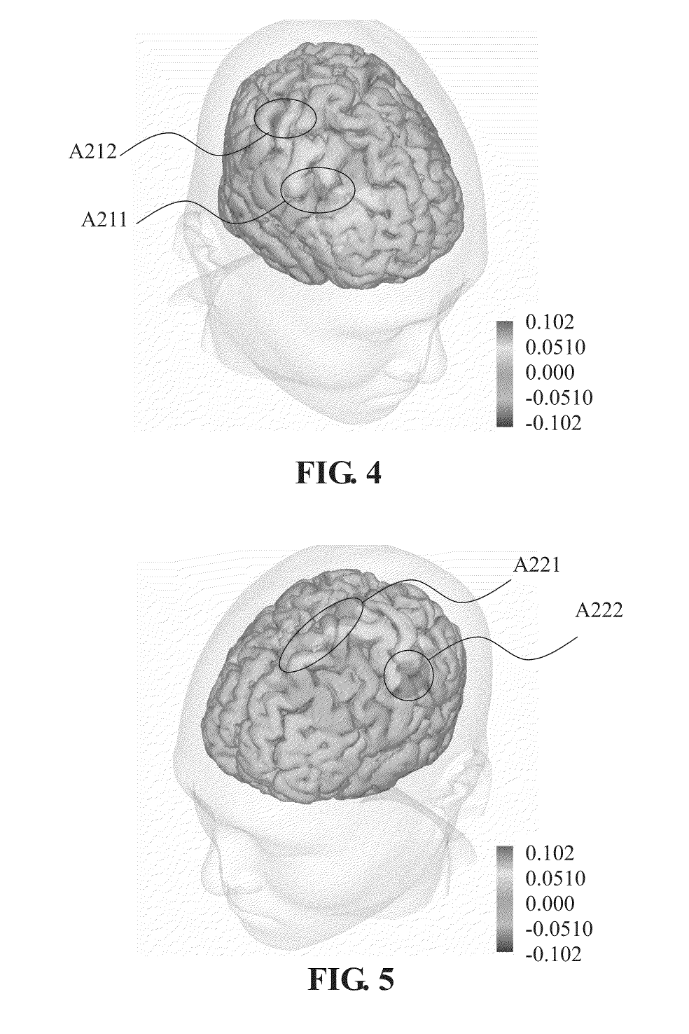

Next, the active region in the tested area A11 where real-time variation in the cerebral blood flow occurs (corresponding to a region with significant increase in the blood flow) can be detected within 3 seconds after the subject's answer (the question is designed so that the correct answer is “no”). This active area can be determined as the negative response area, which can comprise a first negative response area A211 and a second negative response area A212. An example of actual testing results is shown in FIG. 4. Differently colored areas can be used to display variations in the cerebral blood flow. As shown in FIG. 4, an area with significant increase in the cerebral blood flow can be identified as a region with a specific range of significantly close colors (FIG. 4 is black and white drawing that cannot show the color variation). The two active regions encircled in FIG. 4 are the first negative response area A211 and the second negative response area A212. Owing to physiologic differences that may appear in the cortex of each individual, it may possible that two active regions are detected as negative response areas, or only one active region is detected as a single negative response area. In this embodiment, the first negative response area A211 is a major region of determining a negative response, and the second negative response area A212 is responsible for auditory information processing. Thus, the second negative response area A212 can be used for verifying whether the subject truly listens to the question and the final result's confirmation.

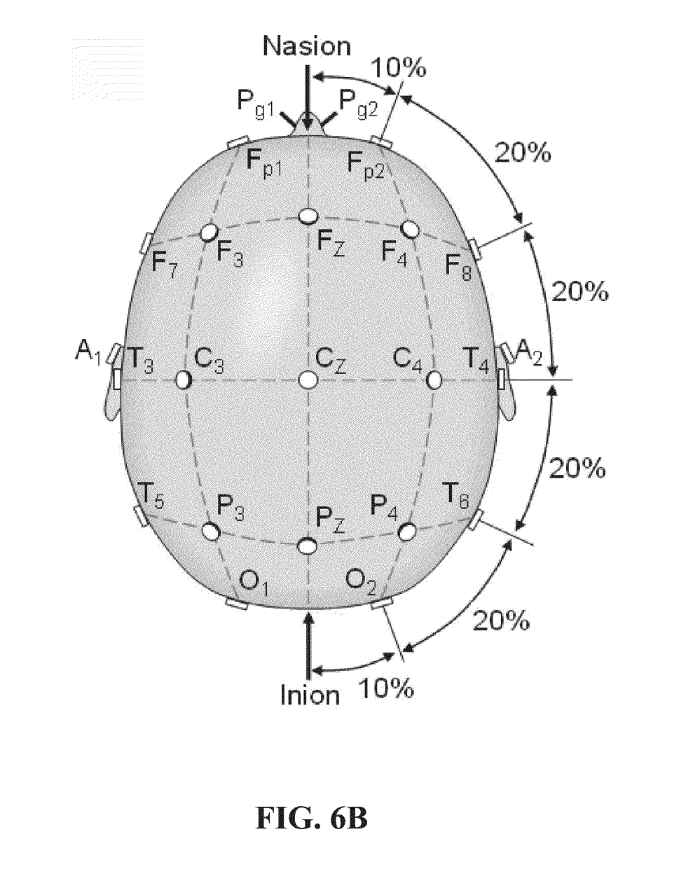

According to international 10-20 system, the negative response area A211, A212 depicted in the present application is located in a region of Cz-Fz-F8-T4 (as shown in FIGS. 6A and 6B) which is also located at right parietal lobe.

### Embodiment 2: Determination of the Affirmative Response Area in the Cortex

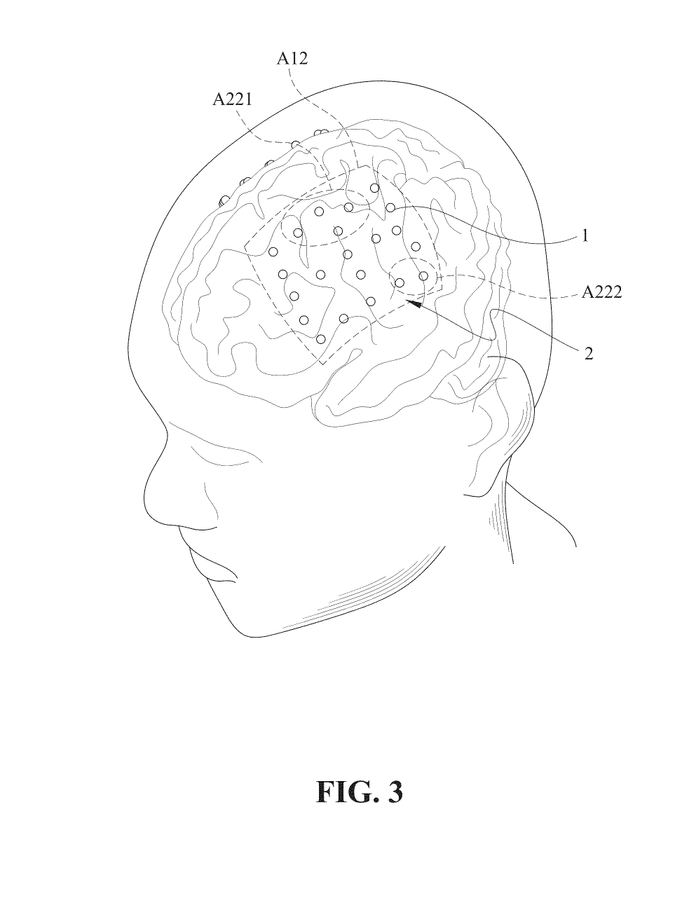

FIG. 3 is a schematic view showing a method of determining an affirmative response area in the cortex. The steps of this method are similar to the aforementioned Embodiment 1. After it is properly set, the near-infrared spectroscopy apparatus for cerebral blood flow detection can be used to generate a test array 2 consisting of a plurality of test points 1 in a tested area A12 of the subject's head.

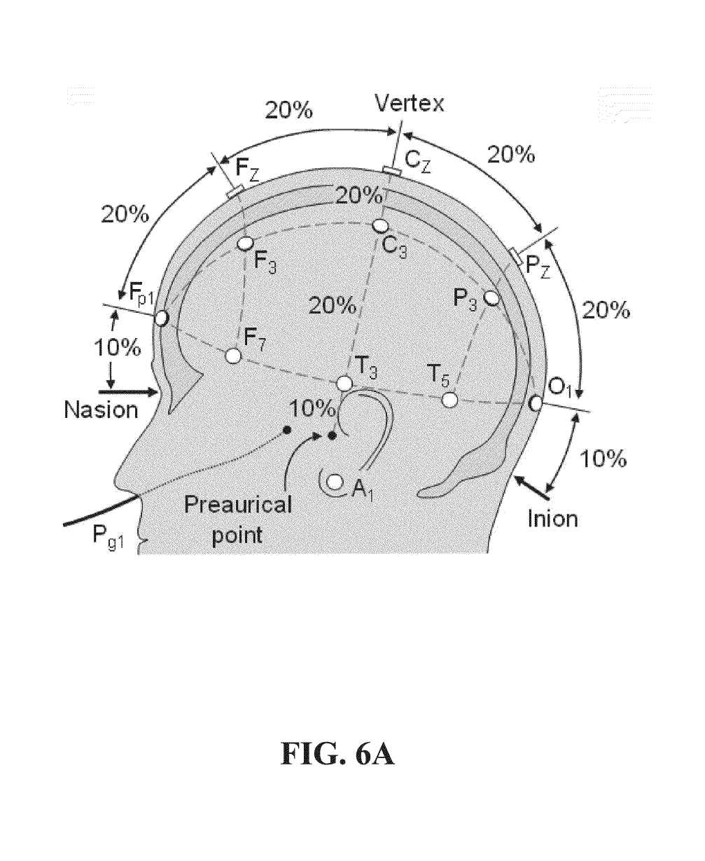

Next, the active region in the tested area A12 where real-time variation in the cerebral blood flow occurs (corresponding to a region with significant increase in the blood flow) can be detected within 3 seconds after the subject's answer (the question is designed so that the correct answer is “yes”). The determined active areas can comprise a first affirmative response area A221 and a second affirmative response area A222. An example of actual testing results is shown in FIG. 5. Differently colored areas can be used to display variations in the cerebral blood flow. As shown in FIG. 5, the two encircled active regions are the first affirmative response area A221 and the second affirmative response area A222 (FIG. 5 is black and white drawing that cannot show the color variation). Like the previous embodiment, owing to physiologic differences that may appear in the cortex of each individual, it may possible that two active regions are detected as affirmative response areas, or only one active region is detected as a single affirmative response area. Similarly, the first affirmative response area A221 is a major region of determining an affirmative response, and the second affirmative response area A222 is also responsible for auditory information processing. Thus, the second affirmative response area A222 can be used for verifying whether the subject truly listens to the question and the final result's confirmation.

According to international 10-20 system, the affirmative response area A221, A222 depicted in the present application is located in a region of Cz-Fz-F7-T3 (as shown in FIGS. 6A and 6B) which is also located at left parietal lobe.

### Conclusion

In conclusion, the present application cannot be achieved through the previous studies. For example, combining the methods and results shown in Tian et al. (2009) and Monti et al. (2010) would generate a contradictory result. The area detected in Tian et al. (2009) is located at PFC, however the depth of penetration by NIRS merely 15-20 mm, thus such a combination of detection by NIRS at a PFC area cannot anticipate the parietal lobe area as disclosed in the present application. In addition, the depth of parahippocampal gyrusdepth detected in Monti et al. (2010) is 50-60 mm from surface, thus a combination of Tian et al. (2009) and Monti et al. (2010) is unachievable.

On the other hand, Tian et al (2009) utilize an experimental procedure of finger pushing to answer questions (see page 125, left column, “4.1 Paradigm”) that is an indirect step for lie detection. Answering by finger tapping actions would induce multiple active regions in cerebral cortex. The supporting evidence for finger tapping can be referred to Suzanne et al. (2008) and Toshimasa et al. (2007). In Suzanne et al. (2008), FIG. 2 and FIG. 3 show that finger tapping causes multiple active brain regions (including the parietal lobe area mentioned in the present application). Furthermore, FIG. 1 in Toshimasa et al. (2007) demonstrates that multiple brain region's activity is also induced after finger tapping (including the regions mentioned in the present application). The finger tapping answering used in Tian et al. (2009) combines the parietal lobe area claimed in the present application would result in multiple induced active regions, which would be difficult to distinguish whether the active regions are induced by finger tapping or purely response to the questions. If we follow the results and methods used in these two references, affirmative response and negative response would be difficult to determine.

The foregoing description is intended to only provide illustrative ways of implementing the present invention, and should not be construed as limitations to the scope of the present invention. While the foregoing is directed to embodiments of the present invention, other and further embodiments of the invention may thus be devised without departing from the basic scope thereof, and the scope thereof is determined by the claims that follow.

What is claimed is:

1. A method of determining negative and affirmative response areas in a cerebral cortex of a human subject under a test, comprising the steps of:
(A) providing a testing apparatus to detect real-time variations in cerebral blood flow;
(B) generating a test array consisting of a plurality of test points in a tested area of the subject's head to detect real-time variations of the cerebral blood flow in the tested area, wherein the tested area approximately corresponds to an affirmative or negative response area in the cortex;
(C) asking a question to the human subject, wherein the question is designed so that an answer for the question is either yes or no; and
(D) determining a precise position of the affirmative or negative response area in the cortex, according to an active region corresponding to the real-time variations of the cerebral blood flow generated in 3 seconds within the tested area after answering;
wherein the testing apparatus is a near-infrared spectroscopy apparatus; asking of step (C) and answering of step (D) are performed in oral form; when the answer is “yes” in step (C), the position determined in step (D) is a affirmative response area; when the answer is “no” in step (C), the position determined in step (D) is a negative response area; the affirmative response area and the negative response area are located at left parietal lobe and right parietal lobe respectively, the affirmative response area is located in a region of Cz-Fz-F7-T3 defined by international 10-20 system, and the negative response area is located in a region of Cz-Fz-F8-T4 defined by international 10-20 system.

2. The method according to claim 1, wherein the affirmative response area comprises a first affirmative response area and a second affirmative response area, and the active region is determined at least one of the first and second affirmative response areas.

3. The method according to claim 2, wherein the second affirmative response area is responsible for auditory information processing.

4. The method according to claim 1, wherein the negative response area comprises a first and a second negative response areas, and the active region is determined at least one of the first and second negative response areas.

5. The method according to claim 4, wherein the second negative response area is responsible for auditory information processing.
